(19) United States
(12) Patent Application Publication
(10) Pub. No.: US 2018/0344507 A1
(43) Pub. Date: Dec. 6, 2018

(54) INTRAORAL DEVICE FOR THE REPOSITIONING AND REHABILITATION OF THE TONGUE

(71) Applicant: Michel ALGLAVE, SAINT-ANDRE (Ile de la Reunion) (FR)

(72) Inventor: Michel ALGLAVE, SAINT-ANDRE (Ile de la Reunion) (FR)

(21) Appl. No.: 15/760,759

(22) PCT Filed: Sep. 14, 2016
(86) PCT No.: PCT/EP2016/071734

§ 371 Date: Aug. 13, 2018

(30) Foreign Application Priority Data
Sep. 17, 2015 (FR) 1558729

(51) Int. Cl. A61F 5/56

(52) U.S. Cl. CPC A61F 5/566; A61C 7/08

(57) ABSTRACT

An intraoral device for the repositioning of the tongue, comprising a support which can be fixed relative to a maxillary dental arch of a patient, wherein the device comprises a blade which is mounted on the support and extends in a longitudinal direction, inscribable within a sagittal plane of the patient, from a posterior end to an anterior end, the blade comprising an elastically deformable portion configured to alternately adopt a rest position, in which the portion positions itself at least partly beyond a bite plane of the patient, and at least one constrained position, in which the portion is deformed in the direction of the bite plane. The device will have a use in all disorders involving poor positioning of the tongue.

### FIELD OF THE INVENTION

The invention relates to an intraoral device for the repositioning and rehabilitation of the tongue.

This invention will have its application in the orthodontics field and more generally in all of the pathologies relating to a poor position of the tongue.

### PRIOR ART

The positioning of the tongue plays an essential role in the growth of the face and in the mouth functions (mastication, phonation, swallowing, etc.). A poor position of the tongue generates many problems. In particular, a bottom position of the tongue, i.e. when the tip of the tongue is placed at the rear of the lower incisors, able to be combined or not with an oral respiration, is the main cause of dento-maxillary dysmorphia, dental malpositions, delays and asymmetries in the growth of the face, lack of oxygenation of the frontal region of the brain, sleep disorders: snoring, hypopnoea, sleep apnoea, temporomandibular joint dysfunction (TMJ) or pronunciation disorders.

There are many devices intended to encourage the tongue to rise in the palate behind the root of the central upper incisors.

These devices are conventionally U-shaped orthodontic trays. Some include a proprioception element, for example a tab, which is positioned on the palate in order to stimulate the tip of the tongue and encourage it to remain at the top. These devices only have a temporary effect.

Other devices include an inclined facet arranged in the mandibular portion making it possible to guide the tongue in the direction of the palate by procuring a support point for it. However, the results are not homogeneous as the tongue has the choice to remain at the bottom on the inclined plane, in a very comfortable position, or to place the tip of the tongue on the palate which is a clearly more tiring position. The patient soon tires and the tongue remains in the bottom position.

None of these devices maintain the tongue in the palate during swallowing which takes place about 2000 times per 24 h.

There is therefore a need to propose a device that allows for good positioning of the tongue while ensuring the sustainability of the suitable position.

### SUMMARY OF THE INVENTION

To this effect, the invention relates to an intraoral device for the positioning of the tongue, comprising a support which can be fixed in position relative to the maxillary dental arch of a patient. Characteristically, the device comprises a blade mounted on the support. The blade extending in a longitudinal direction inscribable within a sagittal plane of the patient from a posterior end to an anterior end. The blade has an elastically deformable portion configured to alternately adopt a rest position in which the portion positions itself beyond a bite plane and a constrained position in which the portion is deformed in the direction of the bite plane.

Advantageously, the blade comprises a segment extending from the rear to the front that has a component oriented in a sagittal plane and moving away from the bite plane.

The blade of the invention makes it possible to divert the tip of the tongue upwards and to the front of the palate while also ensuring its position during swallowings.

This blade is intended to guide the tip of the tongue in top position, as well as constraining it to remain there. If the tongue is not correctly positioned in the top position, the elastically deformable portion is deformed by the tongue, constrained position of the blade which then pushed back the tongue in the good position by returning as such to its rest position. The tip of the tongue rests advantageously on the blade.

The device allows for a correct positioning of the tongue while still ensuring this position over time.

The device according to the invention has the advantage of preventing any other detrimental position of the tongue.

Advantageously, the posterior end of the blade is configured to be applied on the floor of the mouth and as such advantageously delimits a lower compartment entre the floor of the mouth and the blade which is not accessible to the tongue.

### BRIEF DESCRIPTION OF THE FIGURES

The drawings are given by way of examples and do not limit the invention. They form block diagrams intended to facilitate the understanding of the invention and are not necessarily to the scale of the practical applications.

FIG. 1 is a top view of the device according to the invention.

FIG. 2 is a view of the rear face of the device according to the invention.

FIG. 3 is a detailed view according to the section AA of FIG. 2.

FIG. 4 is a bottom view of the device according to the invention.

FIG. 5 is a view of the front face of the device according to the invention.

FIG. 6 is a view according to the section BB of FIG. 5.

FIG. 7 is a detailed view C of FIG. 6 according to an embodiment.

FIGS. 8a to 8c are cross-section views representing the movements of the blade of the device according to the invention when the tongue presses on this blade before positioning itself in top position.

FIGS. 9a and 9b are top views representing the positioning of the tongue on the blade. FIG. 9a corresponds to FIG. 8a and FIG. 9b corresponds to FIG. 8c.

### DETAILED DESCRIPTION

Before beginning a detailed review of embodiments of the invention, hereinafter are announced optional characteristics that can possibly be used in association or alternatively.

It is firstly reminded that the invention concerns a intraoral device intended at repositioning the tongue, comprising a support which can be fixed relative to a maxillary dental arch of a patient, characterised in that it comprises a blade mounted on the support and extending in a longitudinal direction inscribable within a sagittal plane of the patient, from a posterior end to an anterior end, with the blade comprising an elastically deformable portion configured to alternately adopt a rest position in which the portion positions itself at least partly beyond a bite plane of the patient and at least one constrained position in which the portion is deformed in the direction of the bite plane.

Advantageously, the portion has a segment extending from the rear to the front that has a component oriented in a sagittal plane and moving away from the bite plane.

Advantageously, the elastically deformable portion is at least partially curved.

Advantageously, the elastically deformable portion comprises a concavity opposite the bite plane.

Advantageously, the posterior end of the blade is configured to be applied on the floor of the mouth.

Advantageously, the posterior end of the blade is configured to delimit a lower compartment between the floor of the mouth and the blade that is not accessible to the tongue.

Advantageously, the device comprises means for adjusting the portion of the blade.

Advantageously, the means for adjusting include means for providing a sliding of the blade in the grooves formed in the support.

Advantageously, the means for adjusting the portion of the blade provide an adjusting of the length of the blade in such a way as to be atraumatic for the floor of the mouth.

Advantageously, in constrained position, the deformed portion is positioned beyond the bite plane.

Advantageously, in rest position, the elastically deformable portion comprises a surface configured to support the tongue.

Advantageously, the surface configured to support the tongue is smooth.

Advantageously, the blade comprises an anterior portion configured to be concave in the rest position and in constrained position in such a way as to provide for the return to rest position of the blade.

Advantageously, the posterior end of the blade comprises a rounded edge that has a median notch configured to allow the frenulum of the tongue to pass.

Advantageously, a rounded edge of the blade comprises two tabs spaced by the median notch and configured to be applied on the floor of the mouth.

Advantageously, the blade comprises a distal portion extending to the posterior end and having a curvature opposite that of the elastically deformable portion.

Advantageously, the portion has an arched shape in a plane perpendicular to the concavity thereof.

Advantageously, the device comprises at least one front fastening point of the blade on an anterior zone of the support.

Advantageously, the device comprises at least one rear fastening point of the blade on a posterior zone of the support.

Advantageously, between the front and rear fastening points, the blade is free to move.

Advantageously, the device comprises means for fastening the blade to the support on the fastening points.

Advantageously, the blade is removable.

According to a separable aspect, the invention relates to an intraoral device intended at repositioning the tongue, comprising a tray provided with a maxillary support surface and with two symmetrical demi-arches on either side of a median direction and joining in a front zone of the tray, characterised in that it comprises a blade extending from the front zone to the rear end of the two demi-arches, the blade at least partially protruding above the maxillary support surface and comprising a segment extending from the rear to the front that has a component oriented in a sagittal plane and directed beyond the maxillary support surface.

Advantageously, the blade extends from the front zone to the rear end of the two demi-arches and the blade has an elastically deformable portion configured to alternatively have a rest position wherein the portion is positioned at least partially beyond the maxillary support surface and at least one constrained position wherein the portion is deformed in the direction of the maxillary support surface.

In the description which follows and unless mentioned otherwise, symmetrical means the fact that the device of the invention advantageously has a symmetry, either for some parties along a median direction axis which will be explained hereinafter, or along a plane of the sagittal type. The term symmetrical does not mean that slight dimensional or geometrical variations are impossible around a symmetry in the strict sense. In particular, according to the corrections to be made to the morphology of the user, the device can have slight variations between the left and right lateral portions of the invention able to cooperate with the dental arches. The term “sagittal plane” is to be understood in its usual anatomical definition as a plane vertical and perpendicular to the plane seen from the front. Typically, this plane is parallel to an anteroposterior axis of the head and passes through a point located midway between the two eyes. In the framework of the invention, the median direction is included in the sagittal plane, the sagittal plane able to be defined moreover as the plane containing said median direction and substantially perpendicular to the maxillary support surface which will be presented in more detail later. The adjective “frontal” is here used to designate a direction of intersection between a frontal plane and the sagittal plane, i.e. encore generally a vertical direction.

Terms of the type under, underneath, beyond are to be understood from a position relatively to a vertical direction generally according to the thickness of the support, more precisely of the tray.

Dental arch sector means an anatomical portion of the user that can include a bone portion and/or a dental portion on which the device can be applied.

The adjective “maxillary” is used in reference to the upper jaw and the adjective “mandibular” in reference to the lower jaw.

The terms rear and posterior as well as front and anterior are used in a similar way and are to be understood according to a direction falling within the sagittal plane. The front is located on the incisors while the rear is located at the bottom of the oral cavity.

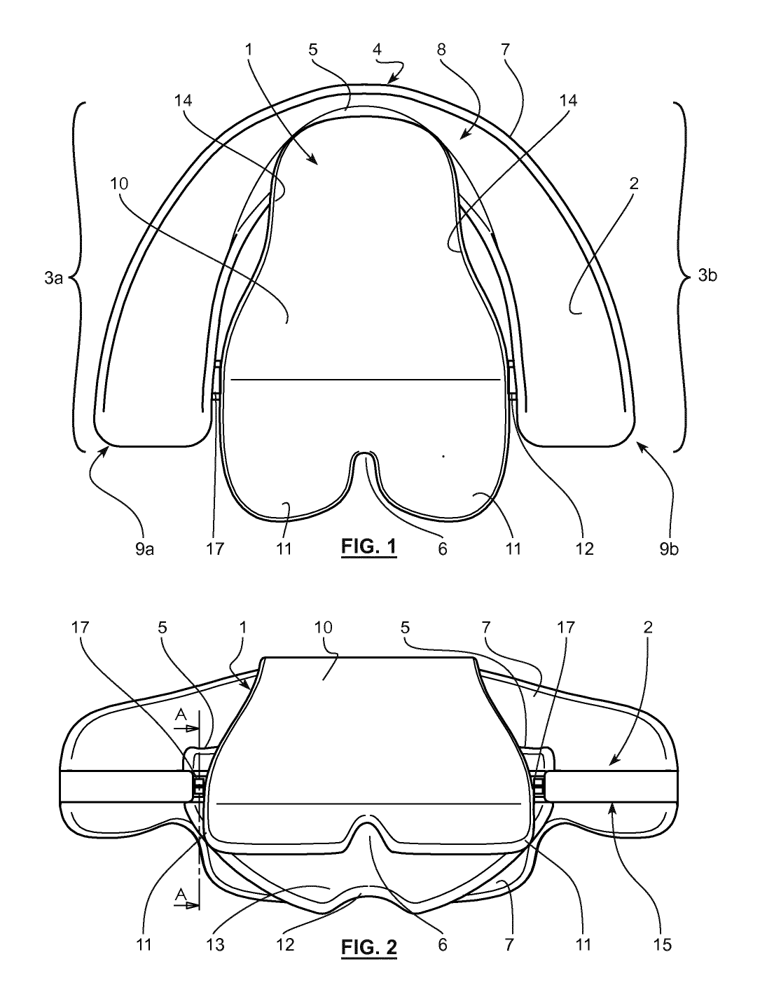

The bite plane is the plane of contact between the maxillary arch and the mandibular arch. In the embodiment shown in the figures, the bite plane is formed by the tray 8 between the maxillary support surface 2 and the mandibular support face 15. The device according to the invention appears in different views in FIGS. 1 to 9. In these figures which show a preferred embodiment, the support is a tray 8 described hereinafter.

The support according to the invention can be fastened to the maxillary arch. “Can be fastened” means that the support is configured to be fastened in position on the maxillary arch. The support can be removable, it is not necessarily embedded on the maxillary arch.

By way of example, the support is a palatal plate also called retainers. The palatal plate is a plate commonly made of resin whereon are fastened hooks and springs intended to cooperate with the teeth of the maxillary arch in order to maintain the support in position.

The blade 1 according to the invention is mounted on the palatal plate. Various types of mounting can be provided; for example, the blade 1 is connected to the palatal plate by lateral fastening points arranged substantially facing the upper lateral incisors or upper canines and on first upper molars. The fastening of the blade 1 on the palatal plate can have an elasticity that facilitates the deformation of the blade 1 and the setting in place of the tongue between the blade 1 and the palatal plate. The blade 1 is advantageously removable.

According to another example, the support is formed by dental rings. The blade 1 is mounted on the rings by lateral fastening points similar to what is described hereinabove for the palatal plate. The blade 1 is advantageously removable.

According to year another embodiment, the support is an arch that can be a contention fastened to the teeth. The blade 1 is mounted on the arch or on the attaching rings of the arch by lateral fastening points similar to what is described hereinabove for the palatal plate. The blade 1 is advantageously removable.

Generally, the blade 1 is more preferably mounted on the support in such a way that once the support is in fastened position on the maxillary arch of the patient, the fastening points of the blade 1 on the support, at least at the front, are advantageously located on upper incisors or possibly canines and on first molars, at the rear of the blade 1, the blade 1 being free between the front and rear fastening points.

The support globally has a shape adapted to the contours of the upper dental arch of the user intended to receive the device.

As with the known devices for positioning the tongue, the device of the invention comprises according to an embodiment a tray 8 allowing for at least one support of the upper jaw. To this effect, the tray 8 comprises a maxillary support surface 2. Advantageously but not exclusively, the maxillary support surface 2 is substantially plane. The maxillary support surface 2 allows for the application of the teeth of the upper dental arch of the user.

Opposite the maxillary support surface 2, the tray 8 preferably comprises a mandibular support face 15 which can be of a configuration substantially identical to the maxillary support surface 2 that is to say preferably flat.

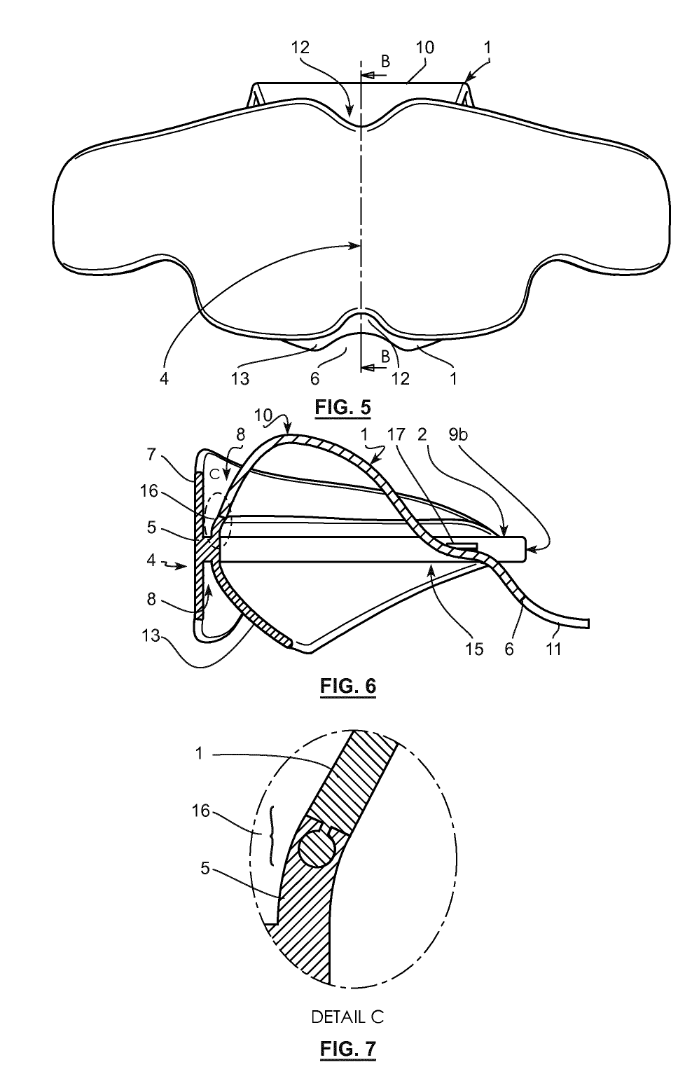

Generally, in order to follow the anatomical contour of the upper dental arch of the user, the tray 8 comprises two demi-arches 3a, 3b, each one placed on a side of the tray 8 around a front zone 4 carrying out the junction between the two symmetrical demi-arches 3a, 3b. The axis of symmetry of the two demi-arches 3a, 3b corresponds to a median direction m of the tray that cuts it into two equal portions. This direction “m” can be seen in particular on the front view, FIG. 5, and corresponds to the cutting plane BB. This median direction “m” corresponds to the intersection between the sagittal plane and a substantially horizontal plane wherein is located at least approximately the maxillary support surface 2.

The two demi-arches 3a, 3b terminate opposite the front zone 4 by posterior ends 9a, 9b corresponding substantially to the last molars of the patient.

It is easily understood that the shape and the dimension of the tray 8 are adapted to the morphology of the user and can therefore be variable. Consequently, the invention can as such be declined into several sizes. The tray 1 is advantageously flexible and therefore able to be elastically deformed as indicated hereinabove and can be made from various biocompatible materials and particularly from silicone.

In the portion presenting the maxillary support surface 2 and the mandibular support face 15, the tray 8 constitutes a bite plane that is inserted between the top arch and the bottom arch. Its typical thickness is about from 2 to 4 millimetres.

The tray 8 comprises a screen 7 and advantageously an upper posterior edge 5. This edge 5 as well as the screen 7 are positioned mainly in the front zone 4 of the tray 8. The upper posterior edge 5 is the edge arranged inside the concavity of the tray 8 while the screen 7 is arranged outside the concavity of the tray 8. The screen 7 is for example intended to maintain the lips at a distance from the teeth. Advantageously the posterior upper edge 5 and the screen 7 follow the convexity of the tray 8. The edge 5 and the screen 7 extend substantially in a plane perpendicular to the plane of the maxillary support surface 2. The edge 5 and the screen 7 form anterior and posterior guides for the maxillary dental arch, in particular for guiding the teeth in the tray 8.

The screen 7 is located in the anterior zone of the device of the invention and constitutes the front portion of the whole. Its shape is advantageously convex. Advantageously, the convexity is executed not only following the planes parallel to the sagittal plane but also following perpendicular planes. The screen 7 has a convexity in the sagittal plane and in the frontal plane. This convexity of the screen 7, more precisely these two convexities allow for a forward movement of the gingivo-labial fold along a sagittal plane. Preferably, the screen 7 comprises an upper facet 8 extending beyond the maxillary support surface 1 and advantageously a lower facet extending beyond the maxillary support surface 2. The screen 7 makes it possible to maintain the lips at a distance from the teeth and as such prevent the lips for exerting detrimental pressure on the teeth.

The screen 7 can be provided with through-openings in such a way as to facilitate the circulation of air.

The screen 7 comprises a curving 12 in the upper facet and a curving 12 in the lower facet of which the shape is preferably configured to respectively arrange a space for the upper tongue frenulum and for the lower tongue frenulum and be atraumatic. The curvings 12 form low points on the sagittal plane.

The screen 7 is intended to be in contact by its external face with the upper and lower lips of the user. Preferably, the upper facet is intended to be in contact with the upper lip Is while the lower fact is intended to be in contact with the lower lip li.

The convex shape of the screen 7 is also intended to be atraumatic for the user who must wear this device over long periods of time, in particular throughout the entire night. The screen 7 is configured to adapt perfectly to the external shape of the upper dental arch.

The screen 7 extends advantageously over practically the entire length of the tray 8. The height of the screen 7 is advantageously decreasing from the front zone 4 of the tray 8 towards the end of the demi-arches 3a-3b.

On the face opposite the maxillary support surface 1, the mandibular support face 15 is advantageously present with at least one edge protruding around said mandibular support face 15. According to a possibility, the device comprises a lower tongue edge 13 able to be housed in the lower palate also called floor of the mouth. The lower tongue edge 13 extending in the continuity of the upper posterior edge 5 of the tray 8 beyond the maxillary support surface 2. The lower tongue edge 13 is formed of a substantially curved layer of which the convexity is oriented towards the tray 8. The lower tongue edge 13 is advantageously of the shape of a sphere or hemisphere sector over an angular portion for example less than a sphere quarter. In addition, the lower tongue edge 13 forms a guide for the positioning of the teeth of the mandibular dental arch and in particular of the incisors in the tray 8 on the mandibular support face 15.

The lower tongue edge 13 extends according to an embodiment over an angular sector plus narrower than the angular sector over which the screen 7 extends. By way of example, the lower tongue edge 13 extends over an angular sector by about 30° of the tray 8.

The device according to the invention characteristically comprises a blade 1. The description hereinafter is given for the embodiment shown in the figures and can be applied without difficulty to any support covered by the invention and in particular those described hereinabove.

The blade 1 extends from the front to the rear of the device and advantageously between the two demi-arches 3a, 3b. The blade 1 is arranged at least partially above the maxillary support surface 2 as such providing a top position of the tongue. Preferably, the blade 1 has a segment extending from the rear to the front that has a component in a sagittal plane and directed upwards, i.e. in a direction extending beyond the maxillary support surface 2. This blade segment 1 is therefore oriented from the rear to the front and from the bottom upwards in such a way as to advantageously guide in an atraumatic way the tip of the tongue to a top position, i.e. the tip of the tongue behind the upper incisors in contact with the palate. This segment advantageously makes it possible to prevent the tongue from being in bottom position, i.e. on floor of the mouth.

The blade 1 is configured to ensure a correct positioning of the tongue, i.e. the tip of the tongue behind upper incisors in contact with the palate. The blade 1 extends from the front zone 4 of the tray preferably from the upper posterior edge 5 to the end of the two demi-arches 3a-3b of the tray 8. Preferably, the blade 1 is fastened to the tray 8 by several fastening points, more precisely advantageously at least one front fastening point on the front zone 4 of the upper posterior edge 5, corresponding to the rear of the roots of the central upper incisors when the device is in place in the mouth of the patient. The blade 1 is advantageously also fixed by at least two rear fastening points on the end of the two demi-arches 3a-3b, more precisely at the rear end of the upper posterior edge 5. The blade 1 is fixed to the bite plane of the tray 8 on tongue faces of the first lower molars when the device is in place in the mouth of the patient. Advantageously, outside of these front and rear fastening points the blade 1 is free. This arrangement facilitates the deformation of the blade as described hereinafter.

The blade 1 comprises at least one elastically deformable portion 10. The blade 1 advantageously has a maximum height between 0.7 and 1.5 cm. The height of the blade 1 extends between its farthest point above the bite plane in a sagittal plane and said bite plane. The portion 10 of the blade 1 is configured to have alternatively a rest position and preferably at least one tension or constrained position. The blade 1, in tension position, is configured to automatically return to the rest position. The blade 1 is referred to as shape memory, in that the deformation thereof is elastic.

Advantageously, regardless of the position of the blade 1, the portion 10 is positioned at least partially above the maxillary support surface 2.

Preferably, the portion 10 comprises a concavity opposite the maxillary support surface 2 in rest position.

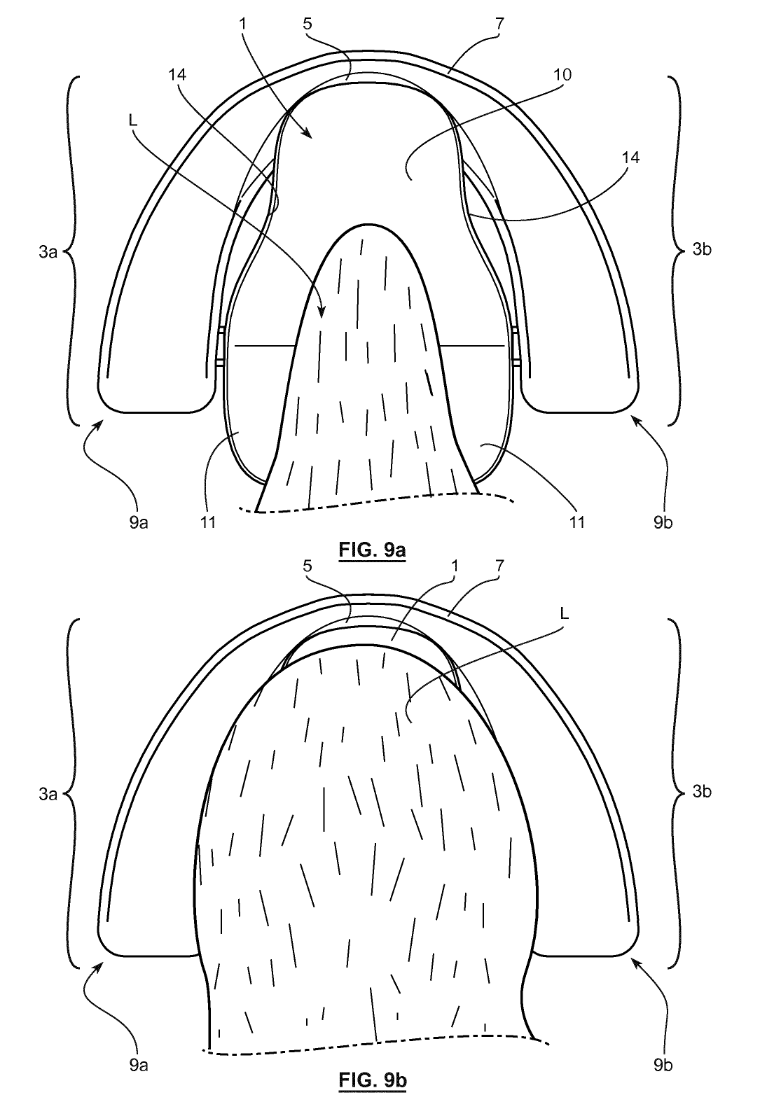

The rest position is the stable position of the blade 1. In the rest position, the concave portion 10 of the blade 1 is positioned at least partially above the maxillary support surface 2. The concavity is directed towards the top of the device i.e. at the opposite of the maxillary support surface 2 beyond which the portion 10 protrudes. The portion 10 can be curved, in particular with an arc of circle. In this rest position, shown in the FIGS. 1 to 7 as well as 8c and 9b, the tongue is in the top position with the tip positioned in contact with the upper palate behind the incisors. The langue is spread out and has a rounded tip as shown in FIG. 9b, the tongue is also flat. The tongue is correctly maintained, even during swallowings.

Advantageously, the portion 10, preferably on the concavity, in rest position of the blade 1 is configured to receive and support the tip of the tongue in high position. The portion 10 advantageously has a surface forming a support plate for a lower zone of the tongue. Preferably, this surface is smooth in such a way as to facilitate the propulsion of the tip of the tongue upwards and towards the front as well as to support the tongue in an atraumatic way.

Advantageously, this surface includes the highest point of said portion 10, i.e. the farthest point above the bite plane.

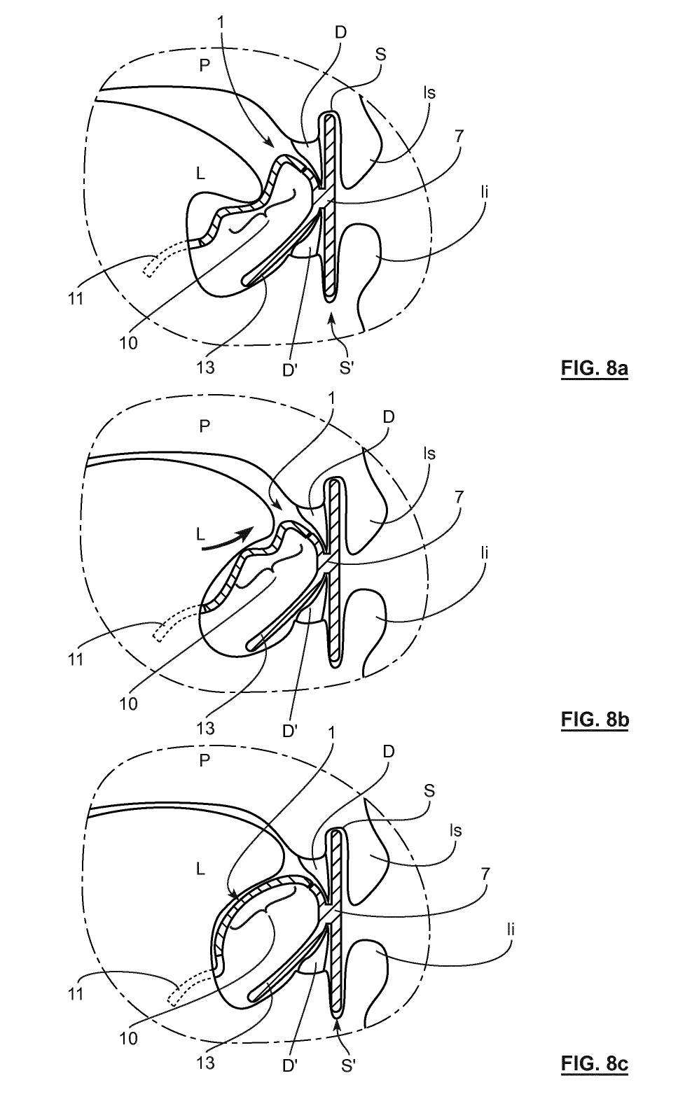

In tension position, also called the constrained position, the blade 1 is in an imbalanced position. The portion 10 is deformed. This blade position 1 is shown in FIGS. 8a, 8b and 9a. According to a possibility shown in FIGS. 8a and 8b, the portion 10 has a convexity opposite the concavity of the portion 10 in rest position. Advantageously, the deformed portion 10 is always positioned above the maxillary support surface 2.

Preferably, the blade 1 comprises an anterior portion, i.e. advantageously arranged between the front fastening point and the concave portion 10 which also has a concavity. This concavity is in the extension of the concavity of the portion 10 in rest position and remains in concave form when the portion 10 is deformed in constrained position. This anterior portion which remains concave regardless of the position of the elastically deformable portion makes it possible to provide this elastic deformation and the automatic resuming of the rest position of blade 1.

Advantageously, the blade 1 forms a partition separating the cavity into two compartments: an upper compartment, close to the palate and a lower compartment, close to the floor of the mouth and blocking the access to the lower compartment.

According to a particularly preferred embodiment, the blade 1 comprises a distal portion extending to the posterior end. The distal portion advantageously has, at least in rest position, a curvature, or a concavity, opposite that of the portion 10. The blade 1 preferably has, at least in rest position, two opposite curvatures. The blade 1 is configured to be deformed as an accordion during a thrust of the portion 10 by the tongue. The two curvatures are either arranged in the extension of each other giving a substantially S-shape of the blade 1, or the blade comprises an inflection in the direction of the bite plane, between the concavity of the portion 10 and the concavity opposite the rear end of the blade 1.

According to an example shown in the figures, the blade 1 is extended beyond rear fastening points located at the rear of the device. Preferably, the extension of the blade makes it possible to provide a contact with the floor of the mouth. The blade 1 advantageously has a rounded edge and a median notch 6 configured to arrange a space for the tongue frenulum and be atraumatic and advantageously forming two tabs 11. The blade 1 is expended beyond fastening points, located at the rear of the device, and below the maxillary support surface 2. The tabs 11 are located under the maxillary support surface 2. The tabs 11, i.e. the atraumatic lower edge of the blade 1, are intended to be in contact with the floor of the mouth in such a way as to prevent the passage of the tongue under the blade 1. Preferably, the bottom position of the tongue is no longer possible because the lower compartment of the oral cavity is no longer accessible.

From the front zone 4 to the rear of the device the blade 1 advantageously comprises the anterior portion, then the elastically deformable portion 10, then the segment oriented from the rear to the front and from the bottom upwards that can possibly be elastically deformable and be a part of the portion 10, then the distal or posterior portion comprising according to a possibility the tabs 11 and advantageously the median notch 6 in order to allow the frenulum of the tongue to pass.

The blade 1 is a flexible blade. More preferably, the thickness of the blade 1 is less than 2 mm, preferably 1 mm.

The thickness of the blade 1 can be varied along a longitudinal axis of the blade 1. For example, the blade 1 can be thinner at the rear end and/or thicker on the portion 10 in such a way as to be atraumatic and contribute to the elastic deformation of the portion 10. The blade 1 can be carried out in various biocompatible materials particularly silicone.

According to a possibility, the blade 1 has an arched shape, preferably in a plane perpendicular to the concavity, and advantageously on the curved portion 10. In this way, the blade 1 has a lesser width at the top of the portion 10, in rest position. The portion 10 advantageously has a narrowing 14 on either sided. This arched shape facilitates the deformation of the blade 1 and the return thereof to rest position.

Generally, the blade 1 is mobile from bottom to top in the tray and advantageously has a width that is less than the space delimited between the two demi-arches 3a, 3b.

According to an embodiment shown in FIGS. 6 and 7, the blade 1 is in part removable. The device comprises means for fastening 16 the blade 1 on the tray 8 and preferably on the upper posterior edge 5. These means of fastening 16 make it possible to fasten the blade 1 and therefore to form a tongue platform such as described hereinabove in order to facilitate the tongue positioning on a base tray 8 for example on a conventional orthodontic appliance.

By way of a non-limiting example, these means for fastening 16 are for example male-female systems such as notches and complementary protrusions on the upper posterior edge 5 such as shown in FIG. 7 and on the blade 1 as well as at the ends 9a, 9b of the two demi-arches 3a-3b and on the blade 1. It is as such possible to form a base portion of the device able to cooperate selectively with one among several blades 8 in particular for a simple adaptation to the different anatomies of users. The fastening is fast and simple.

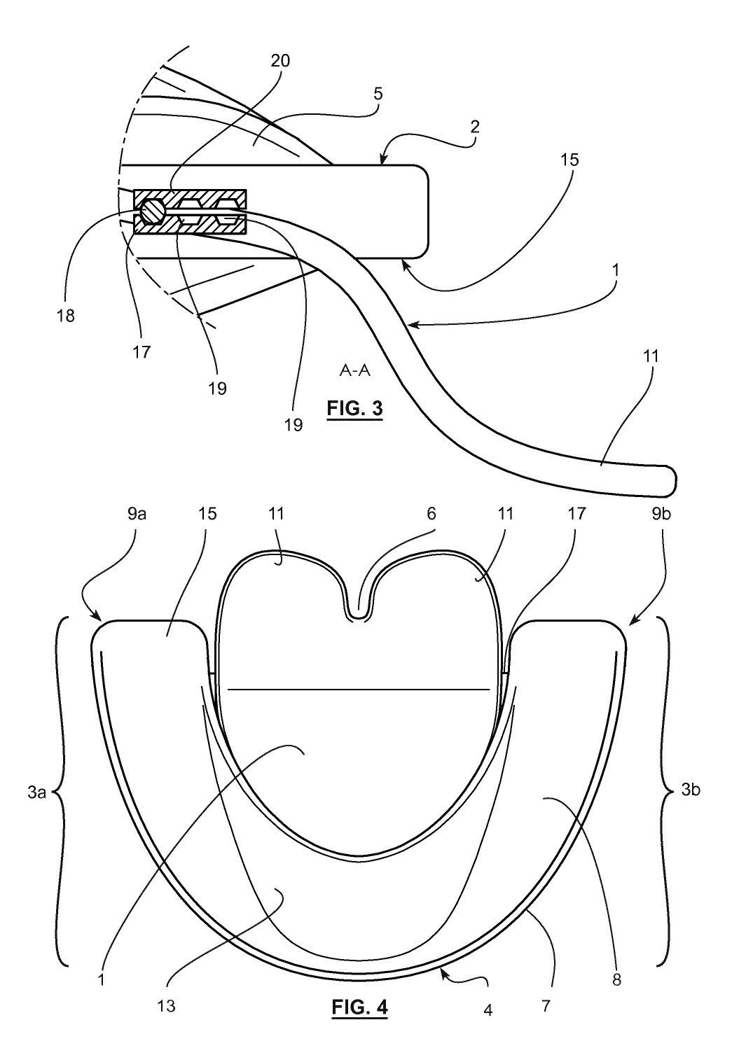

According to an embodiment shown in FIGS. 2 and 3, the device comprises means for adjusting 17 the curved portion 10. These means for adjusting 17 are intended to allow for a modification in the length and/or in the height and/or in the concavity of the curved portion 10 in such a way as to adapt as best as possible to the morphology of each patient. By way of a non-limiting example, the means for adjusting 17 allow for a sliding of the blade 1 in the complementary grooves formed in the tray 8 preferably on the tongue face on roots of the first lower molars. According to another embodiment shown in FIG. 3, the means for adjusting 17 include means of male/female fastening and a slide, such as for example a tenon 18 is formed on the blade 1 cooperating with mortises 19 wherein are fixed the tenon 18. The mortises 19 formed in the tray 8 are connected by a slide 20 wherein the tenon 18 slides.

After the adjusting of the length of the blade 1, the practitioner can cut the lower end of the blade 1, preferably on tabs 11, in such a way as to limit any traumatising friction for the floor of the mouth.

An operating procedure of the invention is described herein after.

The mandible of the user that clamps the teeth is placed in the canal formed by the tray 8 on the maxillary support surface 2. Advantageously, the tray 8 enters the mouth of the user by being contracted transversally between the thumb and the index finger.

Beforehand, the dimensions of the device could have been adjusted or the size suitable to the morphology of the user found and in particular thermoforming of the tray 8 according to the desired transversal dimension according to the size of the palate of the user and by adapting the dimensions of the blade 1.

The tongue is then placed either directly in the top position in contact with the portion 10 of the blade 1 in rest position, or the tongue pushes with its tip downwards on the portion 10 of the blade 1 driving its deformation in tension position. The blade then returns the tip of the tongue upwards and towards the front to return to its ideal rest position and position the tongue in the top position. When the tongue pushes on the blade 1 as shown in FIG. 8a, the latter has a narrow and pointed shape as shown in FIG. 9a.

The transversal thrust of the tongue and its correct positioning in the top position makes it possible to re-establish a normal growth of the face.

The device according to the invention is indicated for rehabilitating the position of the tongue at rest, during swallowing, during the day as well as at night. At night, the tongue is thrust against the palate and will not tend to be released obstructing the pharynx and causing the uvula to vibrate generating snoring.

### REFERENCES

1. Blade

2. Maxillary support face

3a-3b demi-arch

4. Front zone of the tray

5. Upper posterior edge

6. Median notch

7. Screen

8. Tray

9a-9b posterior ends

10. Portion

11. Tab

12. Curving

13. Lower tongue edge

14. Narrowing

15. Mandibular support face

16. Means for fastening

17. Means for adjusting the curved portion

18. Tenon

19. Mortise

20. Slide

M. Median direction

Ls. Upper lip

Li. Lower lip

L. Tongue

S. Upper gingivo-labial fold

S′. Lower gingivo-labial fold

P. Palate

D. Tooth—upper central incisor

D′. Tooth—lower central incisor

1. An intraoral device for the repositioning of the tongue, comprising a support which can be fixed relative to a maxillary dental arch of a patient, wherein the device comprises a blade mounted on the support and extending in a longitudinal direction inscribable within a sagittal plane of the patient, from a posterior end to an anterior end, with the blade comprising an elastically deformable portion configured to alternately adopt a rest position in which the portion positions itself at least partly beyond a bite plane of the patient and at least one constrained position in which the portion is deformed in the direction of the bite plane.

2. The device according to claim 1, wherein the portion has a segment extending from the rear to the front that has a component oriented in a sagittal plane and moving away from the bite plane.

3. The device according to claim 1, wherein the elastically deformable portion is at least partially curved.

4. The device according to claim 1, wherein the elastically deformable portion comprises a concavity opposite the bite plane.

5. The device according to claim 1, wherein the posterior end of the blade is configured to be applied on the floor of the mouth.

6. The device according to claim 1, wherein the posterior end of the blade is configured to delimit a lower compartment between the floor of the mouth and the blade that is not accessible to the tongue.

7. The device according to claim 1, comprising adjusting elements of the portion of the blade.

8. The device according to claim 1, wherein the adjusting elements provide a sliding of the blade in grooves formed in the support.

9. The device according to claim 7, wherein the adjusting elements of the portion of the blade provide an adjusting of the length of the blade in such a way as to be atraumatic for the floor of the mouth.

10. The device according to claim 1, wherein in constrained position, the deformed portion is positioned beyond the bite plane.

11. The device according to claim 1, wherein in rest position, the elastically deformable portion comprises a surface configured to support the tongue.

12. The device according to claim 11, wherein the surface configured to support the tongue is smooth.

13. The device according to claim 1, wherein the blade comprises an anterior portion configured to be concave in the rest position and in constrained position in such a way as to provide for the return to rest position of the blade.

14. The device according to claim 1, wherein the posterior end of the blade comprises a rounded edge that has a median notch configured to allow the frenulum of the tongue to pass.

15. The device according to claim 14, wherein the rounded edge of the blade comprises two tabs spaced by the median notch and configured to be applied on the floor of the mouth.

16. The device according to claim 1, wherein the blade comprises a distal portion extending to the posterior end and having a curvature opposite that of the elastically deformable portion.

17. The device according to claim 16, wherein the portion has an arched shape in a plane perpendicular to the concavity thereof.

18. The device according to claim 1, comprising at least one front fastening point of the blade on an anterior zone of the support.

19. The device according to claim 1, comprising at least one rear fastening point of the blade on a posterior zone of the support.

20. The device according to claim 18, wherein between the front and rear fastening points, the blade is free to move.

21. The device according to claim 1, comprising fastening elements of the blade to the support on the fastening points.

22. The device according to claim 1, wherein the blade is removable.
